(19) United States
(12) Plant Patent Application Publication
(10) Pub. No.: US 2008/0184439 P1
(43) Pub. Date: Jul. 31, 2008

(54) Agonis flexuosa plant named 'Jedda's Dream'

(76) Inventor: Jacqueline Ann Koppman, Huskisson (AU)

Correspondence Address: Jacqueline Koppman;c/o Carolyn Sluis, 6500 Donlon Road (PO702), Somis, CA, 93066, US

(21) Appl. No.: 11/889,531

(22) Filed: Aug. 14, 2007

(30) Foreign Application Priority Data
Aug. 15, 2006 (AU) 2006/222

(51) Int. Cl. A01H 5/00

(52) U.S. Cl. PLT/356

(57) ABSTRACT

‘Jedda's Dream’ is a distinctive variety of Agonis flexuosa, which is characterized by its bushy plant growth habit, strong basal branching, short plant height, short internode length, red purple immature stem color, gray purple immature leaf color, overall red purple foliage appearance and short leaf length.

### LATIN NAME OF THE GENUS AND SPECIES

The Latin name of the novel variety disclosed herein is Agonis flexuosa (Willd.) Sweet.

### VARIETY DENOMINATION

The inventive variety of Agonis flexuosa disclosed herein has been given the variety denomination ‘Jedda's Dream’.

### BACKGROUND OF THE INVENTION

The present invention relates to a new and distinct variety of evergreen perennial Agonis flexuosa, which has been named ‘Jedda's Dream’. Agonis flexuosa is a small to medium Australian native tree to 10 m high that is used as an ornamental tree in landscape and amenity horticulture. Agonis flexuosa has an upright to semi-weeping plant growth habit with light to medium basal branching and a medium plant height. Stems have medium internode length and immature stem color is variable from red purple to yellow green to grayed orange. Flowers are white and appear in spring. Immature leaf color is yellow green and matures to a darker green color. Overall foliage color is green. Leaf length is medium. Leaf fragrance is aromatic when crushed similar to a peppermint aroma.

An application for plant breeders' rights for ‘Jedda's Dream’ has been lodged with the Australian Plant Breeders Rights Office, and was accepted on 15 Aug. 2006 (under Application No. 2006/222).

Parentage: The cultivar ‘Jedda's Dream’ was selected as a whole plant mutation in 1999 in Falls Creek, New South Wales, Australia, during vegetative propagation of cultivated Agonis flexuosa variety ‘Jervis Bay Afterdark’ (unpatented, Australian Plant Breeders Rights application no. 1997/225). The parent form is characterized by an upright- semi-weeping plant growth habit with light to medium basal branching, tall plant height, medium internode length, red purple immature stem color, gray purple immature leaf color and medium leaf length. The overall foliage appearance of the parent form is a red purple color. Selection criteria for ‘Jedda's Dream’ were short plant height and bushy plant growth habit.

Asexual reproduction. The new variety ‘Jedda's Dream’ was first asexually propagated by vegetative cuttings in the state of New South Wales, Australia in 2001. The distinctive characteristics of cultivar ‘Jedda's Dream’ have remained stable and true to type through successive cycles of asexual propagation.

### SUMMARY OF THE INVENTION

‘Jedda's Dream’ is a distinctive variety of Agonis flexuosa, which is characterized by its bushy plant growth habit, strong basal branching, short plant height, short internode length, red purple immature stem color, gray purple immature leaf color, overall red purple foliage appearance and short leaf length.

### BRIEF DESCRIPTION OF THE DRAWINGS

FIG. 1 shows an Agonis flexuosa ‘Jedda's Dream’ plant at approximately 18-months of age in a 200 mm pot. The plant was propagated in a greenhouse and was transferred to the field at six months and grown out in full sun.

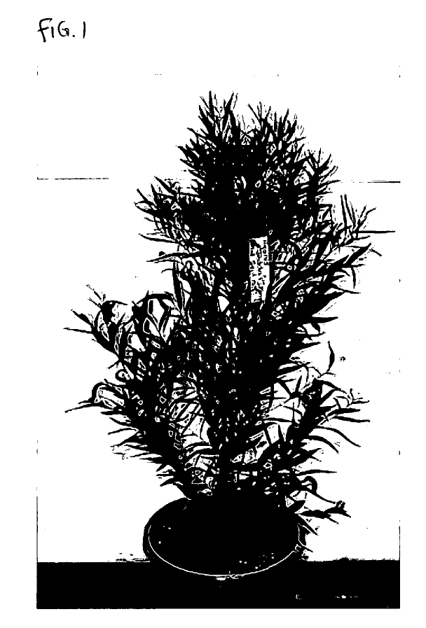

FIG. 2 shows the colors and habit of ‘Jedda's Dream’ (at left) versus the parent variety ‘Jervis Bay Afterdark’.

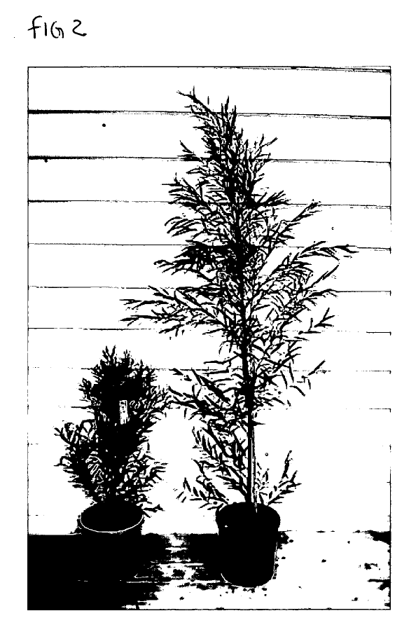

FIG. 3 shows the colors of leaves of the new growth of ‘Jedda's Dream’.

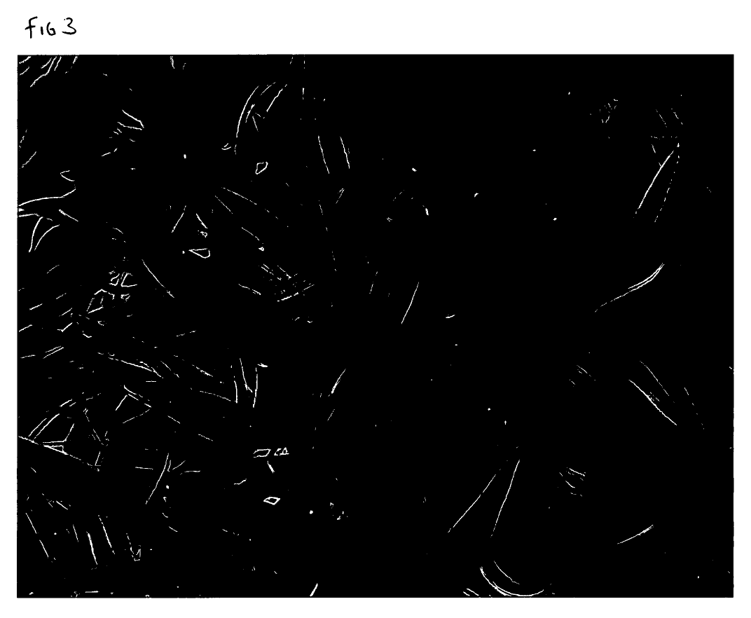

FIG. 4 shows the shoot tip habit including branching, stem color and leaf shape of ‘Jedda's Dream’.

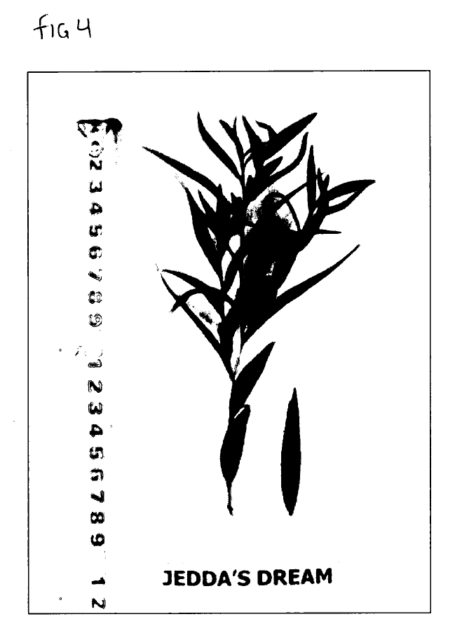

### DETAILED BOTANICAL DESCRIPTION OF THE VARIETY

The following is a detailed botanical description of a new and distinct variety of Agonis flexuosa known as ‘Jedda's Dream’ based upon observations of 18-month old plants grown in 200 mm nursery pots in full sun in open beds in Falls Creek, New South Wales, Australia during 2005-2006 (as described below in connection with Table 1). Plant observations and descriptions were taken in winter 2006.

Those skilled in the art will appreciate that certain characteristics will vary with older or, conversely, with younger plants. ‘Jedda's Dream’ has not been observed under all possible environmental conditions. Where dimensions, sizes, colors and other characteristics are given, it is to be understood that such characteristics are approximations or averages set forth as accurately as practicable. The phenotype of the variety may differ from the descriptions set forth herein with variations in environmental, climactic and cultural conditions. Color notations are based on The Royal Horticultural Society Colour Chart, The Royal Horticultural Society, London, 1995 edition. ‘Jedda's Dream’ is an evergreen perennial Agonis flexuosa. ‘Jedda's Dream’ is a bushy tree with an upright growth habit and a medium growth rate growing to 2-3 metres high in 34 years. ‘Jedda's Dream’ has a large number of branches appearing basally on the main stem'. ‘Jedda's Dream’ has a short plant height with short internodes on its stems creating a compact appearance for the plant. ‘Jedda's Dream’ has a red purple to grayed purple immature stem color which matures to a grayed orange color. ‘Jedda's Dream’ has a yellow green unexpanded leaf color changing to a grayed purple immature leaf color and then maturing to a darker yellow green color. ‘Jedda's Dream’ has a lanceolate leaf shape with occasional distortion to a falcate shape. Leaf apex shape is acute and margin is entire with inconspicuous pubescence. Immature leaves can be recurved along the longitudinal axis and mature to a flattened profile. Leaf length of ‘Jedda's Dream’ is short. Occasional leaf twisting is present. The surface of the mature leaf upper side of ‘Jedda's Dream’ has a weakly glossy appearance, pinnate venation, absence of pubescence and the midrib coloration matched the leaf blade. The surface of the mature leaf blade lower side of ‘Jedda's Dream’ has a weakly glossy appearance, pinnate venation, absence of pubescence and the midrib coloration is a grayed purple contrasting to the yellow green leaf blade. Leaf arrangement is alternate and distichous. This combination of characteristics is unusual for Agonis flexuosa, as these plants usually have an upright to semi-weeping plant growth habit with light to medium basal branching and a medium plant height with a longer internode length. A botanical description of ‘Jedda's Dream’ and comparisons with the parent form of Agonis flexuosa are provided below.

### TECHNICAL DESCRIPTION OF THE VARIETY

Plant characteristics: Growth habit bushy, compact and upright height short (2-3 meters high in 34 years), number of branches appearing at stem base is large, overall foliage appearance is a red purple color.

Stems: strongly branching, new stem has a roughened surface texture with 2 opposing wings and slight pubescence, internode length is short (range is 10-12 mm for new shoots, 10-22 mm for secondary stems and 20-25 mm for the main stem on an 18 month old plant), color of young stem red purple (RHS 59A) to grayed purple (RHS 187A), gradually maturing to grayed orange (RHS 164A-165B). The old bark on old stems and the trunk has an overall grayed brown appearance composed of a mixture of grayed orange (RHS 164A) and grayed brown (ca RHS 199D).

Leaves: arrangement is alternate and distichous, attitude horizontal to semi-erect once mature with the upper side facing the stem, attachment sessile, length short (range 55 to 65 mm for a mature leaf), width narrow (range 10-11 mm), shape lanceolate with occasional distortion to a falcate shape, apex acute, cross section flat, immature leaves can be recurved along the longitudinal axis and mature to a flattened profile, margin entire with inconspicuous pubescence, leaf twisting is infrequently present, surface texture of the mature leaf upper side has weak glossiness, venation pinnate and corresponding to leaf color, variegation absent, unfolding leaf color yellow green (RHS 146D), new expanding leaf upper side color brown (RHS 200A when viewed over a white background) or grayed purple (RHS187A) when light transmits through the leaf tissue, mature leaf color darker than yellow green (RHS 147A), mature leaf lower side color corresponds to upper side, upper side midrib color corresponds to leaf blade (darker than yellow green (RHS 147A), lower side midrib color grayed purple (RHS 187A).

Flowers and fruiting: Flowering characteristics of ‘Jedda's Dream’ have yet to be recorded. Typical flower color is white and flower season is spring.

Cold and Heat Tolerance: ‘Jedda's Dream’ has not yet been observed under all conditions.

‘Jedda's Dream’ is heat tolerant. It adapted well to the high heat conditions greater than 40 degrees Celsius during summer in New South Wales, Australia without any noticeable damage. It has survived frosts to zero degrees Celsius. Testing for environmental tolerances is ongoing.

Drought tolerance: Good drought tolerance typical of the species once established. ‘Jedda's Dream’ has not yet been observed under all conditions. Testing for environmental tolerances is ongoing.

Pest Resistance: No known pests.

Cultural conditions: ‘Jedda's Dream’ can tolerate low nutrient conditions; it does not like continually wet soil conditions, but can tolerate well-draining sandy soils to very heavy clay soils. The species has been recorded as being tolerant of limestone areas.

### COMPARISONS WITH OTHER AGONIS FLEXUOSA

‘Jedda's Dream’ is a more attractive ornamental tree as compared with the parent ‘Jervis Bay Afterdark’. ‘Jedda's Dream’ is characterized by its bushy plant growth habit, strong basal branching, short plant height, short internode length and short leaf length as compared with ‘Jervis Bay Afterdark’. The appearance of ‘Jedda's Dream’ is short, compact and bushy, whereas ‘Jervis Bay Afterdark’ an upright to semi-weeping growth habit, a taller plant with less basal branching (see Table 1). Agonis flexuosa (unpatented, wild type) typically has an upright to semi-weeping growth habit, a medium-tall height with green foliage whereas ‘Jedda's Dream’ has an overall red purple foliage appearance. The variety ‘Burgundy’ has an upright-weeping plant growth habit, longer internode length, longer leaf length and less basal branching than ‘Jedda's Dream’.

A comparative trial of Agonis cultivar ‘Jedda's Dream’ with Agonis cultivars ‘Jervis Bay Afterdark’ and ‘Burgundy’ is currently being carried out in Falls Creek, New South Wales, Australia. The initial data are presented in Table 1 below.

TABLE 1

Agonis Variety

| | ‘Jedda's | ‘Jervis Bay | |
|---|---|---|---|
| Characteristic | Dream’ | Afterdark’ | ‘Burgundy’ |
| Plant: growth habit | upright, | upright to | upright to |
| | bushy | semi- | semi- |
| | | weeping | weeping |
| Plant: height | short | medium | short |
| Plant: basal branching | strong | medium | medium |
| Stem: internode length | short | medium | medium |
| Leaf: length | short | medium | medium |

That which is claimed is:

1. A new and distinct variety of Agonis flexuosa plant named ‘Jedda's Dream’, substantially as described and illustrated herein.
